(19) United States
(12) Patent Application Publication
(10) Pub. No.: US 2018/0243113 A1
(43) Pub. Date: Aug. 30, 2018

(54) Stent

(71) Applicant: Daniel Ezra Walzman, Bergenfield, NJ (US)

(72) Inventor: Daniel Ezra Walzman, Bergenfield, NJ (US)

(21) Appl. No.: 15/732,544

(22) Filed: Nov. 22, 2017

Related U.S. Application Data
(60) Provisional application No. 62/497,851, filed on Dec. 5, 2016.

(51) Int. Cl. A61F 2/852; A61F 2/91; A61L 31/12; A61L 31/14; A61L 31/02; A61L 31/04

(52) U.S. Cl. CPC A61F 2/852; A61F 2/91; A61L 31/125; A61F 2002/823; A61L 31/022; A61L 31/04; A61L 31/146

(57) ABSTRACT

The present invention teach an overlapping covered stents with uncovered segments capable of effectively diverting blood flow away from an aneurysm or fistula while allowing blood to flow to its normal vascular territory of healthy tissue distal to the targeted treatment area and still resulting in blood stasis and thrombus formation inside the aneurysm or fistula.

### BACKGROUND OF THE INVENTION

### Field of the Invention

The present disclosure relates to medical devices used to treat aneurysms and fistulas within unhealthy blood vessels, and more particularly, relates to endovascular devices.

### Background Art

The prior art teaches the use of a number devices to treat aneurysms. A common blood vessel difficulty is the persistent blood flow in the aneurysm sac extrinsic to the endograft. In fact, this is the most common complication after endovascular aneurysm repair (EVAR). Such endoleaks are ameliorated by a number of means. For example, Walzman's 15/732,147 and 15/732,365 teach the use of hydrogel to prevent endoleaks.

The prior art also teaches endovascular coiling as a minimally invasive technique performed to prevent blood from flowing into an aneurysm. This treatment results in the coil inducing embolization (clotting) of the aneurysm, which prevents blood from flowing into the aneurysm, which in turn, prevents subarachnoid hemorrhage. Endovascular coiling however, may result in procedural complications include thromboembolism, cerebral embolization, aneurysm perforation, parent artery occlusion, coil migration, arterial dissection, and others. The prior art also teaches stent-assisted coiling. The stent-assisted coiling also has some of the same short comings related to stent placement and placing a stent in the parent artery requires prolonged use of anti-platelet agents to reduce the risk of thrombosis based stenosis within the stent. There is no covered neuro-stent currently available in the United States. The U.S. Food and Drug Administration (FDA) has examined and tested such covered neuro-stents but none has “FDA approval,” which means that the FDA has not decided the benefits of the previous versions of covered neuro-stents outweigh the potential risks for the item's planned use. A potentially significant use of covered neuro-stents is for the treatment of fistulas, particularly for Carotid cavernous fistula (CCF) which is an abnormal communication between the cavernous sinus and the carotid arterial system.

Other treatment of Aneurysms include surgical clipping. Surgical clipping of an intracranial aneurysm, which involves the application of a clip across the neck of the aneurysm. This treatment has several short comings including that it requires an open operation and physical manipulation of the brain.

Additionally, prior art teaches the use of flow diversion devices to divert flow away from the aneurysm by placing a mesh stent or a structure similar to a stent, on the aneurysm neck along the parent artery. The use of these devices allow for thrombus formation inside the aneurysm. However, increased technical complications can develop following the deployment of flow diverters. Additionally, because they do not completely block flow, they are not effective in the treatment of fistulas and ruptured vessel. Similarly, there is currently no effective treatment of a vessel sparing iatrogenic rupture of an intracranial artery. Current treatment requires closing the ruptured artery with coils and/or liquid embolics to stop the bleeding, usually with significant resulting morbidity from ischemic injury to that arterial territory. Furthermore, when treating aneurysms with these devices, the aneurysm thromboses over time, a lag period, and is not immediately cured. This leaves the patient at risk of aneurysmal rupture during lag period.

A need exists for an endovascular device capable of endovascular intervention for immediate cure of select intracranial aneurysm or a fistula such as a carotid cavernous fistula, while ameliorating the difficulties and shortcomings associated with the currently available technologies. The present invention teach a covered stent device capable of effectively diverting blood flow away from an aneurysm, fistula, or ruptured vessel while allowing blood to flow to healthy tissue distal to the targeted treatment area and still resulting in blood stasis and thrombus formation inside the aneurysm or fistula.

### SUMMARY OF THE INVENTION

The current invention is an improvement over prior art flow-impeding stent technology. Unlike the flow-impeding hydrogel stent of the prior art, the current invention does not rely on complex advanced hydrogel technologies to achieve flow arrest. The ability to effectively use hydrogel in this way remains an unproven technology, and may not be feasible. (Although in select cases the current invention may also employ supplemental hydrogel to help seal the stents to each other and/or to the vessel wall, and to prevent endoleaks). Furthermore, whereas with the hydrogel flow impeding stent in the prior art there may be a short lag in the stent becoming covered in vivo, during which time there can be continued bleeding of a ruptured vessel and/or aneurysm, and continued flow through a fistula, such flow arrest will be immediate upon proper deployment of the device(s) described herein. More particularly, a stent system is disclosed that requires at least two overlapping stents (one inside the other) to be covered fully. The prior art teaches a completely covered stent is possible. The prior art has also attempted to apply covered stents to neuro applications and failed. Said attempts to apply covered stents to neuro applications resulted in devices which were too stiff and insufficiently flexible to allow for intracranial delivery. The present invention overcomes this shortcoming by combining alternating zones of covered and uncovered stent. The uncovered zones allow articulation/bending of the stent along curves—both when it is being delivered and when it is deployed.

The need to use a single stent device or two (or more) overlapping stent devices depends on the situation. For example, if a CC fistula with a very small whole—say 1 mm, requires treatment and a stent with alternating 2.5 mm covered and 1.5 mm uncovered zones is used, then a single device would be sufficient. However, when a fistula with a 5 mm hole requires treatment then two (dual) overlapping stents should be used in a staggered deployment so there is a longer covered zone and the coverage would better conform to the area requiring treatment.

The stent covering can be polytetrafluoroethylene (PTFE) or textile fabric such as Dacron, polyester, pericardium, hydrogel, combinations thereof, and/or others. In the preferred embodiment, all the stents used in a single application use the same covering material. In an alternative embodiment the covering material may differ from stent to stent and from stent segment to stent segment. For example, the inner stent might have added hydrogel on inside for adherence and endoleak prevention with the vessel wall, and on the outside for adherence and endoleak prevention with the outer stent, whereas the inner stent might only have the supplemental hydrogel on the inside to prevent endoleaks between the inner stent and the outer one, as well as the inner stent and the vessel wall wherever the inner stent covers an uncovered segment of the outer stent (in some embodiments of the present invention they may be the same). In some embodiments of the present invention alternating materials from stent segment to stent segment, such as combining PTFE and pericardium or hydrogel.

There are alternating regions along the axis or length of covered and non-covered first stent, following a desired or specified pattern. A second stent bears a corresponding but opposite pattern of covered and uncovered to fill in the gaps of coverage, except for small zones of coverage overlap or gap. When the present invention uses two stents, they need not be mirror images of each other. In the preferred embodiment of the present invention, while the stents are identical, they are deployed in a staggered fashion so that the covered segments of the inner stent cover completely all of the uncovered segments of the first outer stent within the desired covered zone. In addition, there is slight overlap of the covered segments of both stents. The purpose of the overlap is to allow some room for landing error (for example, the stent may move slightly as it expands; if it is off by 1 mm, the intended overlap will still allow full coverage with the second stent). Additionally, either or both stents may have additional hydrogel on any/all parts that can further seal the overlapped covered parts of the two (or more) stents, as well as to seal the covering material to the vessel wall. There may optionally be an inner lining of hydrogel to minimize thrombogenicity and the need for anti-platelets as well.

The current invention has certain additional, optional features, many of which were disclosed in a recent, issued stent patent: resheathable, detachable, electrolytic, thermal, hydrostatic, chemical or other. It may be self-expanding or balloon expanding, open cell or closed cell or other variant or combination; and/or braided, laser cut, or other. It should be noted that since the current invention has both re-sheathing and detachable features, the user of the current invention is able to correct the position if said user is not happy with the location of the present invention. More particularly, said unhappy user need only re-sheath the stent and reposition, and then detach after said user is satisfied with its location. In the preferred embodiment the stents have radio-opaque markers delineating the boundaries of all covered segments.

The present invention is a simple design and is mechanical in nature. Said design may be fabricated from two nearly identical elements. The primary difference between the elements is the diameter of each; thus, fabrication efforts may be halved compared to other overlapping stents.

### DESCRIPTION OF THE DRAWINGS

FIGS. 1A, 1B and 1C are side planar views of three segments of the current inventions and the relative positions of the inner segment, outer segment, and overlapping configuration. FIGS. 1A, 1B and 1C also depict sheathed zones and unsheathed zones disposed such that a covered region and an opening at an intended location are created when overlapped.

FIG. 1A illustrates a side planar view depicting an inner-stent segment with intermittent sheathing along the elongated axis.

FIG. 1B illustrates a side planar view the current invention, depicting an outer-stent segment with sheathing disposed along the elongated axis corresponding to uncovered areas of the inner stent of FIG. 1A.

FIG. 1C illustrates a side planar view of the overlapping stent segments of the current invention, depicting designated openings where sheathing is absent.

FIG. 2. illustrates a lateral view of the overlapping stents of the current invention disposed in place between a posterior communicating artery and internal carotid artery at the target cavernous carotid aneurysm, further showing an open segment allowing blood flow to an ophthalmic artery.

### DETAILED DESCRIPTION OF THE INVENTION

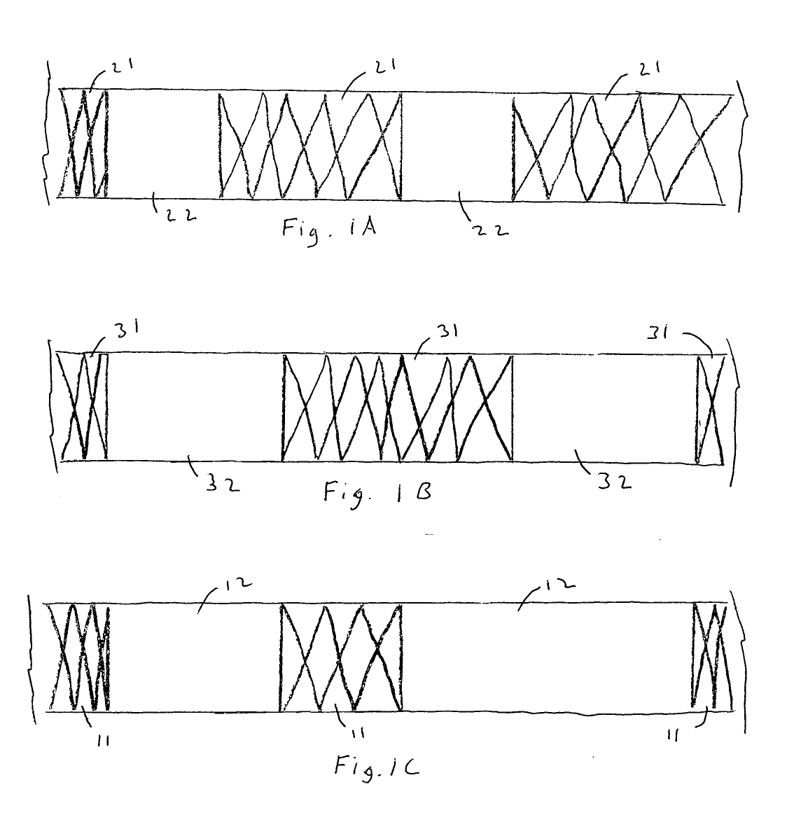

Referring now to FIG. 1, the present invention (10) is composed of at least two corresponding, cylindrically shaped stent bodies (20, 30). Now referring to FIG. 1A, the present invention includes at least one cylindrically shaped stent body (20) composed of covered (22) and uncovered (21) zones. Now referring to FIG. 1B, the present invention also includes at least a second cylindrically shaped body is also composed of covered (32) and uncovered (31) zones. The diameter of said second cylindrical stent body (30) is dimensioned such that said first cylindrical stent body (20) may be inserted snugly therethrough following placement of said second stent body (30) at a target aneurysm or other designated location within a the lumen of a blood vessel.

Referring to FIG. 1C, when first cylindrically shaped body (20) is inserted inside second cylindrically shaped body (30), covered (22, 32) and uncovered (21, 31) zones overlap. Whenever two uncovered zones (21, 31) coincide as shown in FIG. 1C, an uncovered zone (11) is present. Similarly, when two covered zones (22, 32) coincide, a fully covered zone (12) is present. However, when either cylindrically shaped body (20) or cylindrically shaped bodies (30) have a covered zone coinciding with an uncovered zone, then the present invention will still present a covered zone.

A preferred embodiment of the current invention comprises a first cylindrical stent body (20) having alternating 3.0 mm covered (22) and 2.0 mm uncovered (21) segments in cover zone. A second stent body (30) is also disposed with alternating areas of 3.0 mm covered (32) and 2.0 mm uncovered (31), but is deployed in a staggered fashion, to allow for a slight overlap of the covered zones. Said second or outer cylindrical stent body (30) further includes a 6 mm uncovered zone (31) at each end, and a 12 mm “covered” center zone (32).

After said second stent (30) is deployed, said first stent (20) is deployed within the second stent (30) in an alignment so that within the covered zones the covered segments of the inner stent (20) cover the uncovered segments of the outer stent (30), so that there is a net effect of complete continuous coverage within the desired coverage zone.

The edges of the covered zones and each covered segment all have radiopaque markers (not shown) to line up under fluoroscopy. Covered zones can also optionally have adhered hydrogel (not shown) at the edges of coverage. When applied, hydrogel in the second stent (30) preferably faces inside and outside of cylindrical body (30) wall; hydrogel in the first or inner stent body (20) optionally faces inward only. This configuration helps to seal the overlapping covered zones with each other, and with the vessel wall. Other optional configurations of hydrogel may be employed as well.

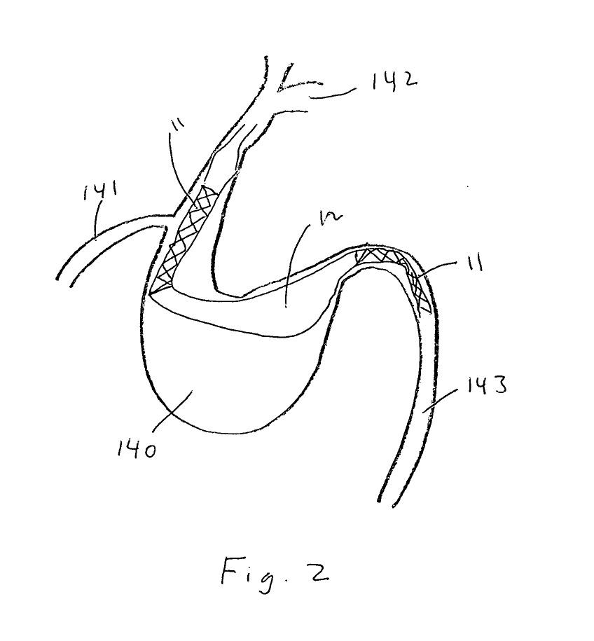

The segments of non-covered stent will allow the stent flexibility to take any necessary turns for delivery and/or deployment. FIG. 2 shows the placement of the overlapped cylindrical stent (10) of the current invention comprised of the inner cylindrical stent body (20) within outer stent body (30) disposed place at target cavernous carotid aneurysm (140) between a posterior communicating artery (142) and internal carotid artery (143). Alternating covered (32) and uncovered (31) segments on the outer or overlapping cylindrical stent body (30) correspond with covered (22) and uncovered (21) segments on the inner cylindrical stent body (20), such that where either the inner stent (20) or outer stent (30) body has a covered segment, or both the inner stent (20) and outer stent (30) have corresponding covered segments (22, 32), the same area is covered. In that case, either inner stent body (20) or outer stent body (30) provides for coverage.

Only where both the inner stent (20) and outer stent (30) have uncovered covered segments (22, 32) at the same location, is an opening present. FIG. 2 illustrates such an opening (11) in the present invention disposed at the juncture of ophthalmic artery (141) proximal to aneurysm (140) such that blood flows freely in to ophthalmic artery (141) but is blocked from aneurysm (140).

The present invention is preferentially for neuro medical difficulties (to treat fistulas, select aneurysms, vessel ruptures and other anomalies), but may be applied to cardiac and peripheral medical difficulties as well. The present invention can be applied in vessels and/or other tubular organs, for example in the ureter and/or bile duct.

Other prior covered stents have proven to be somewhat rigid, and not flexible enough for delivery and deployment in tortuous vasculature, such as that typically encountered for intracranial endovascular delivery and deployment. Unlike the structure of other prior covered stents, the present invention has alternating segments of uncovered stent within the desired covered zone—allowing the stent to flex at the uncovered segments. But in order to cover those uncovered segments within the desired covered zone the new system employs a second (minimum) overlapping stent within the first, which also has alternating covered and uncovered segments for flexibility. But when deploying the first stent body (20) within the second stent body (30), the respective radiopaque markers (not shown), which can optionally have different shapes on the outer stent and the second inner stent, are aligned so that the covered zones of the second inner stent cover the uncovered zones (in the target area of the target covered zone) of the first stent, so that together the two (or more) stents supply a zone or zones of complete coverage.

Furthermore, unlike applicant Walzman's prior invention, the present invention does not rely primarily on hydrogel as the covering agent (although supplemental hydrogel is still optionally used to enhance the seal between the covered regions on the two (or more) overlapping stents, and to prevent endoleaks).

Although the invention has been described in detail in the foregoing embodiments for the purpose of illustration, it is to be understood that such detail is solely for that purpose, and that variations can be made therein by those skilled in the art without departing from the spirit and scope of the invention, except as it may be described by the following claims.

I claim:

1. An intravascular stent device comprising:
(a) a first cylindrical stent body and second cylindrical stent body;
(b) wherein said first cylindrical stent body fits snugly inside of said second cylindrical stent body
(c) wherein said first cylindrical stent body and said second cylindrical stent body maintain a similar orientation;
(d) wherein said first cylindrical stent body and said second cylindrical stent body are made of a solid composite of a polymer; and
(e) wherein said composite is perforated with holes of sufficient size, location and orientation to allow blood to pass from said interior first cylindrical stent body to the exterior of said second cylindrical stent body.

2. A stent according to claim 1 wherein the stent body has a metal surface.

3. A stent according to claim 1 wherein the stent body has a polymeric surface.

4. A stent according to claim 1 wherein the solid composite includes a plurality of perforated layers.

5. A stent according to claim 1 wherein additional cylindrical stent bodies are place insider of said first cylindrical stent body.

6. A stent according to claim 1 wherein additional cylindrical stent bodies are placed outside of said second cylindrical stent body.
